(19) United States
(12) Patent Application Publication
(10) Pub. No.: US 2023/0235306 A1
(43) Pub. Date: Jul. 27, 2023

(54) ARGONAUTE PROTEIN FROM EUKARYOTES AND APPLICATION THEREOF

(71) Applicant: Hubei University, Wuhan (CN)

(72) Inventors: Lixin Ma, Wuhan (CN); Ruyi He, Wuhan (CN); Baotong Sun, Wuhan (CN); Fei Wang, Wuhan (CN); Yaping Wang, Wuhan (CN); Zhongchen Li, Wuhan (CN); Guangbo Yan, Wuhan (CN)

(21) Appl. No.: 18/172,482

(22) Filed: Feb. 22, 2023

Related U.S. Application Data
(63) Continuation of application No. PCT/CN2022/119596, filed on Sep. 19, 2022.

(30) Foreign Application Priority Data
Jan. 24, 2022 (CN) 202210082114X

(51) Int. Cl. C12N 9/22; C12N 15/11; C12N 15/113; C12N 15/90

(52) U.S. Cl. CPC C12N 9/22; C12N 15/11; C12N 15/90; C12N 15/111; C12N 15/113; C12N 2310/14; C12N 2310/20; C12N 2800/80

(57) ABSTRACT

An Argonaute protein from eukaryotes and an application thereof are provided. An amino acid sequence of the Argonaute protein is shown in SEQ ID NO: 1 or has at least 50% sequence identity with the sequence shown in SEQ ID NO: 1. The specific cleavage activity of the eukaryotic Argonaute protein on DNA is first proved, and an experimental proof for the study of interaction between the eukaryotic Argonaute protein and DNA is provided. In addition, polypeptides, nucleic acids, expression vectors, compositions, kits, and methods used therein can carry out site-specific operation on intracellular and extracellular genetic materials and can be effectively applied in many fields of biotechnology, providing a new tool for gene editing, modification, and molecular detection of Argonaute polypeptides based on eukaryotic sources.

### TECHNICAL FIELD

The disclosure relates to the field of molecular biotechnologies, more particular to an Argonaute protein from eukaryotes and an application thereof.

### STATEMENT REGARDING SEQUENCE LISTING

The sequence listing associated with this application is provided in text format in lieu of a paper copy and is hereby incorporated by reference into the specification. The name of the XML file containing the sequence listing is 23002JHG-USP1-SL.xml. The XML file is 74,331 bytes; was created on Feb. 9, 2023; contains no new matter; and is being submitted electronically via EFS-Web.

### BACKROUND

Eukaryotic Argonaute (abbreviated as eAgo) plays a key role in the RNA interference (RNAi) pathway of eukaryotes and is the main functional core of RNA-induced silencing complex (RISC). It can combine with a single-stranded RNA molecule as a small guide specifically recognizing a complementary RNA target, or directly cleave the target through the inherent nuclease activity of some eAgo enzymes, or recruit other silencing proteins to act on the target to inhibit transcription. Therefore, the eAgos can regulate gene expression at transcriptional level, protect host from RNA virus invasion, and maintain genomic integrity by reducing transposon mobility. Recent studies have also shown that some AGO proteins can regulate gene expression through other mechanisms in addition to their role in the typical RNAi pathway. Previous studies found that AGO1 exists in the nucleus, which means that AGO1 may also have important functions in the nucleus, but the specific mechanism has not been explained. At present, most studies only report the in vitro specific cleavage activities of eAgo for RNA, such as human Argonaute 2 (hAgo2) and Kluyveromyces polysporus Argonaute (KpAgo), and no literature reports the specific cleavage activity of eAgo for DNA.

For a long time, people have paid extensive attention to the important role of eAgo in the RNAi pathway, and eAgo with high RNase activity has been found in higher animals and plants, and yeast cells, but the interaction between eAgo and DNA has not been explored in detail. Although some studies have found that eAgo protein can target RNA with single-stranded DNA as a guide, its DNA cleavage activity has not been found. In addition, with regard to the application of Argonaute protein in gene editing, because the eAgo protein from higher animals and plants may participate in the RNAi pathway in the receptor cell and cannot achieve effective gene editing, there is no use of the eAgo protein for gene editing at present.

### SUMMARY

In view of this, the disclosure aims to provide an Argonaute protein from eukaryotes, and the Argonaute protein has DNA cleavage activity is found, which is expected to be applied to gene editing in mammalian cells.

In a first aspect, the disclosure provides an Argonaute protein derived from eukaryotes (hereinafter referred to as eAgo protein or eAgo). The protein is any one of:

A1) a protein with an amino acid sequence as shown in SEQ ID NO: 1; and A2) a protein with at least 50%, at least 80%, at least 90% or at least 95% sequence identity with the amino acid sequence as shown in SEQ ID NO: 1 and the same function as the protein with the amino acid sequence as shown in SEQ ID NO: 1.

Specifically, the protein with the amino acid sequence as shown in SEQ ID NO: 1 derived from Thermomycetes thermophilus eukaryotes and designated as TteAgo protein (also referred to as eAgo protein).

In an embodiment, the eAgo protein is an artificially synthesized protein or an extracted natural protein.

In an embodiment, the eAgo protein has a nuclease activity at a temperature in a range of 10~60° C. Further, the eAgo protein has the nuclease activity at a temperature in a range of 25-55° C. Furthermore, the eAgo protein has the nuclease activity at 37° C.

In an embodiment, the eAgo protein loses the nuclease activity through mutation.

In a second aspect, the disclosure provides a nucleic acid molecule encoding the eAgo protein.

Specifically, the nucleic acid molecule is one selected from a group consisting of:

B1) a DNA molecule with a nucleotide sequence as shown in SEQ ID NO: 3; B2) a DNA molecule hybridizing with the DNA molecule shown in SEQ ID NO.3 under a strict condition and encoding the eAgo protein; and B3) a DNA molecule having at least 50%, at least 80%, at least 90% or at least 95% sequence identity with the nucleotide sequence of the DNA molecule defined in one of the B1) and the B2) and encoding the eAgo protein.

In a third aspect, the disclosure provides an eAgo complex, which is formed by a combination of the eAgo protein and a guide molecule, and the guide molecule is a guide single stranded DNA (ssDNA) or a guide RNA (also referred to as gRNA).

In an embodiment, the guide molecule is one selected from a group consisting of a 5′-terminal phosphorylated guide RNA, a 5′-terminal hydroxylated guide RNA, a 5′-terminal phosphorylated guide ssDNA, and a 5′-terminal hydroxylated guide ssDNA.

In an embodiment, a length of the guide ssDNA is 12 to 40 nucleotides. Further, the length of the guide ssDNA is 12 to 30 nucleotides. Furthermore, the length of the guide ssDNA is 15 to 20 nucleotides, such as 16, 17 or 18 nucleotides.

In a fourth aspect, the disclosure provides the eAgo or the eAgo complex, when the eAgo or the eAgo complex has the nuclease activity, it can specifically cleave a target nucleic acid in vivo or in vitro, and the target nucleic acid is a target RNA (also referred to as tRNA) or a target DNA (also referred to as tDNA).

It should be noted that the target RNA has no advanced structure, or has an advanced structure, or is a double-stranded RNA, or is an RNA transcribed in vitro, or is a viral genome RNA, or is a messenger RNA (i.e., mRNA), or other RNA in the cell. The target DNA is a synthetic single-stranded DNA or a double-stranded DNA; or it can be cellular genomic DNA or other DNA in the cell.

In an embodiment, the eAgo protein or the eAgo complex has the nuclease activity in a solution of divalent metal cations, and the divalent metal cations are at least one selected from a group consisting of Fe²⁺, Co²⁺, Ni²⁺, Cu²⁺, Zn²⁺, Mg²⁺, Mn²⁺, and Ca²⁺.

In an embodiment, the divalent metal cations are at least one of Mn²⁺ and Mg²⁺.

In an embodiment, the divalent metal cations are Mn²⁺.

In an embodiment, the nuclease activity of the eAgo protein or the eAgo complex has single-base and/or double-base specificity.

Specifically, applications of the eAgo protein or the eAgo complex in specific cleavage of the target RNA or the target DNA in vivo or in vitro can be divided into the following four types.

(1) The eAgo protein or the eAgo complex cleaves a target RNA in vitro, and its application process can be as follows. The eAgo protein is mixed with a guide molecule to form an eAgo complex, and the guide molecule is an ssDNA or an RNA. The eAgo complex is in contact with the target RNA containing a nucleotide sequence substantially complementary to a sequence of the guide molecule, and the eAgo-guide complex cleaves the target RNA at a specific site.

(2) The eAgo protein or the eAgo complex cleaves a target DNA in vitro, and its application process can be as follows. The eAgo protein is mixed with a guide RNA to form an eAgo complex. The eAgo complex is in contact with the target DNA containing a nucleotide sequence substantially complementary to a sequence of the guide RNA. The eAgo-guide complex cleaves the target DNA at a specific location.

(3) The eAgo protein or the eAgo complex cleaves a target RNA in cells, and its application process can be as follows. The eAgo protein is mixed with a guide molecule to form an eAgo complex, and the guide molecule is a guide ssDNA or a guide RNA. The eAgo complex is transferred into the cells through transformation, transfection or transduction, and an RNA (i.e., target RNA) in the cell contains a nucleotide sequence substantially complementary to a sequence of the guide molecule.

(4) The eAgo protein or the eAgo complex cleaves a target DNA in cells, and its application process can be as follows. The eAgo protein is mixed with a guide RNA to form an eAgo complex. The eAgo complex is transferred into the cells through transformation, transfection or transduction, and a DNA (i.e., target DNA) in the cell contains a nucleotide sequence substantially complementary to a sequence of the guide RNA.

It should be noted that the target RNA or the target DNA contains the nucleotide sequence complementary to the sequence of the guide RNA or the guide ssDNA, which means that the guide molecule is either completely complementary to the sequence of the same length contained in the target RNA or the target DNA, or there are many mismatches (usually isolated or continuous), and the number of mismatches may be 1, 2, 3, 4 or 5, etc.

In an embodiment, the target RNA or the target DNA contains a nucleotide sequence complementary to at least 12 bases of the sequence of the guide RNA or the guide ssDNA.

In an embodiment, when the target RNA or the target DNA is cleaved in the cell, the cell is an in situ cell.

In a fifth aspect, the disclosure provides an expression vector, which contains the nucleic acid molecule provided in the second aspect.

In a sixth aspect, the disclosure provides an application of the expression vector for site-specific modification of cells in genetic materials.

In an embodiment, the application method is: introducing the expression vector into the cell, and simultaneously or not simultaneously introducing one or more guide RNAs, or introducing one or more guide DNAs; and expressing one or more of the eAgo proteins in the cell.

In an embodiment, multiple eAgo proteins are encoded by one expression vector.

In an embodiment, the expression vector is contained in a virus vector. Specifically, the viral vector is a lentiviral vector or a retroviral vector.

In an embodiment, the cell is an isolated cell.

In an embodiment, the cell is an in situ cell, which can be a living tissue, an organ, or an animal cell including from a human.

In an embodiment, the cell is a eukaryotic cell.

In a seventh aspect, the disclosure provides a kit, which includes the eAgo protein, at least one guide ssDNA and/or guide RNA.

In an eighth aspect, the disclosure provides another kit, which includes the expression vector, at least one guide ssDNA and/or guide RNA.

It should be noted that the selection of the guide ssDNA or the guide RNA in the kit refers to the eAgo complex.

The beneficial effects of the disclosure are as follows.

The disclosure provides eukaryotic-derived Argonaute (i.e., eAgo) polypeptide that can cleave the target nucleotide sequence under the guidance of nucleic acid chain, and proves that TteAgo from Thermothelomyces thermophilus has not only the activity of cleaving RNA but also the activity of cleaving DNA, and puts forward the application potential of eAgos in DNA targeted editing.

The disclosure provides the expression vector containing nucleic acid encoding the polypeptide, and the composition, the kit and the application method for cleaving and editing target nucleic acid in a sequence-specific manner. The polypeptide, nucleic acid, expression vector, composition, kit and method of the disclosure can carry out site-specific modification of intracellular and extracellular genetic materials, so that they can be effectively applied to many fields of biotechnology, such as nucleic acid detection, gene editing and gene modification, and provide a new tool for gene editing, modification and molecular detection of the Argonaute polypeptide based on eukaryotic sources.

The protein provided by the disclosure has binding activity to the guide RNA and the guide ssDNA, and has the nuclease activity to the target RNA and the target DNA. Therefore, when the guide RNA or the guide ssDNA with most of the paired sequence of the target RNA or the target DNA binds to the eAgo protein to form the eAgo-guide complex, and when the eAgo-guide complex binds to the target RNA or the target DNA, site-specific cleavage of the target RNA or the target DNA will occur. The site specificity can be regulated by selecting the guide RNA or the guide ssDNA with a specific nucleotide sequence.

The eAgo protein used in the disclosure can specifically cleave the target RNA and/or the target DNA using the guide RNA and/or the guide DNA with a length of 16-18 nucleotide bp (nt, also referred to as nucleotide base pair), and particularly has high activity when using the ssDNA as a guide to cleave RNA, while the guide DNA has a short synthesis period and a low price compared with RNA, so that the cost can be greatly saved.

In addition, the eAgo protein used in the disclosure does not rely on the special motif near the target site to identify and bind the target, and the guide DNA is convenient to design without considering the site limitation.

The eAgo protein used in the disclosure has strong cleavage activity, which is strictly dependent on the complementary pairing of the guide and the target to play the cleavage activity. There is no non-specific “incidental cleavage” activity of clustered regularly interspaced short palindromic repeats (CRISPR)-related protein, and the specificity is better. In addition, the active site of the eAgo protein can be mutated to obtain an eAgo protein that has completely lost its cleavage activity and can fuse with other effector proteins, further expanding its application.

### BRIEF DESCRIPTION OF DRAWINGS

In order to illustrate technical solutions of embodiments of the disclosure more clearly, the following will briefly introduce the drawings required to be used in the embodiments of the disclosure. Apparently, the drawings described below are only some embodiments of the disclosure. For those skilled in the art, other drawings can be obtained from these drawings without creative work.

FIG. 1 illustrates a schematic diagram of an evolutionary tree of some characterized Argonaute (Ago) proteins provided by the disclosure.

FIG. 2 illustrates a schematic sequence alignment diagram of fourteen characterized Ago proteins provided by the disclosure. Specifically, amino acid sequences of the fourteen characterized Ago proteins are shown in SEQ ID NO: 5-20 respectively.

FIG. 3 illustrates a sodium dodecyl sulfate-polyacrylamide gel electrophoresis (SDS-PAGE) diagram of TteAgo protein according to an embodiment 1, in which lane 1 represents a total protein, lane 2 represents a broken bacteria supernatant, lane 3 represents a 200 millimoles per liter (mM) imidazole eluant, lane 4 and lane 5 represent agarose beads after immunity protein (Im7) incubation, and lane 6 and lane 7 represent the supernatant after 3C protease digestion.

FIGS. 4A-4B illustrate schematic diagrams of a guide RNA (shown in SEQ ID NO: 21), a guide DNA (shown in SEQ ID NO: 23), a target RNA (shown in SEQ ID NO: 22) and a target DNA (shown in SEQ ID NO: 25) used for testing according to an embodiment 2. In addition, cleavage products M1 and M2 are respectively shown in SEQ ID NO: 24 and SEQ ID NO: 26.

FIGS. 4C-4D illustrate urea/polyacrylamide gel electrophoresis diagrams of products of the TteAgo protein cleaving the target RNA and the target DNA according to the embodiment 2.

FIG. 5A illustrates a urea/polyacrylamide gel electrophoresis diagram of products of the TteAgo protein cleaving the target RNA mediated by different lengths of guide RNA according to an embodiment 3.

FIG. 5B illustrates a urea/polyacrylamide gel electrophoresis diagram of products of the TteAgo protein cleaving the target RNA mediated by different lengths of guide DNA according to the embodiment 3.

FIG. 5C illustrates a urea/polyacrylamide gel electrophoresis diagram of products of the TteAgo protein cleaving the target DNA mediated by different lengths of guide RNA according to the embodiment 3.

FIGS. 6A-6F illustrate urea/polyacrylamide gel electrophoretic diagrams of products of the target RNA or the target DNA cut by the TteAgo protein guided under conditions of different metal ions according to an embodiment 4.

FIGS. 7A-7F illustrates urea/polyacrylamide gel electrophoresis diagrams of products of the TteAgo protein cleaving the target RNA and the target DNA mediated by a guide molecule under different ion concentrations of Mn²⁺ or Mg²⁺ according to the embodiment 4.

FIGS. 8A-8D illustrate urea/polyacrylamide gel electrophoresis diagrams of products of the TteAgo protein cleaving the target RNA and the target DNA mediated by the guide molecule under different temperature conditions according to an embodiment 5.

FIG. 9A illustrates a schematic diagram of the guide RNA for single-base and double-base mutations according to an embodiment 6.

FIG. 9B illustrates a schematic diagram of the guide DNA for a single-base mutation according to the embodiment 6.

FIGS. 10A-10C illustrate urea/polyacrylamide gel electrophoresis diagrams of products of the TteAgo protein cleaving the target RNA or the target DNA mediated by the guide RNA after the single-base and double-base mutations and the guide DNA after the single-base mutation according to the embodiment 6.

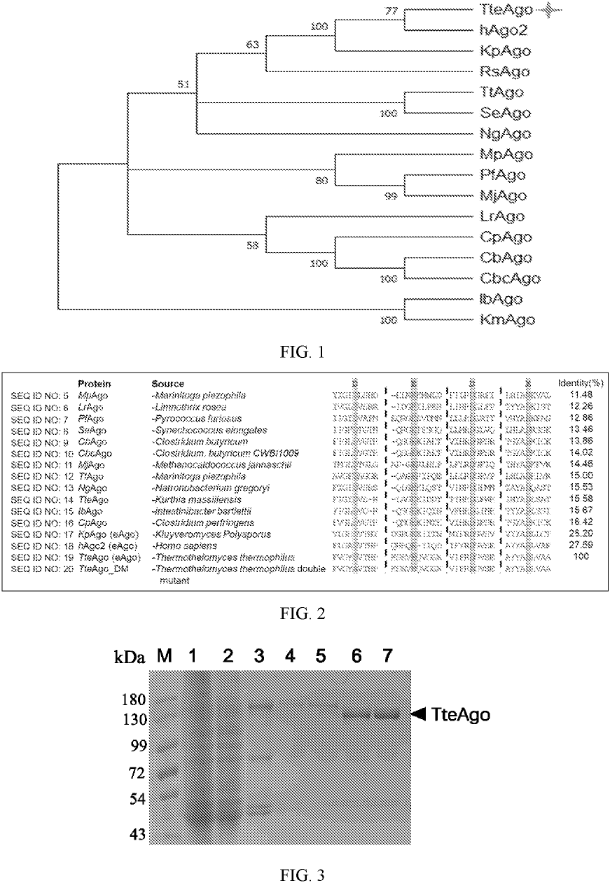

### DETAILED DESCRIPTION OF EMBODIMENTS

In order to make purposes, technical solutions and advantages of the disclosure clearer, the disclosure will be further described in detail in combination with embodiments and drawings. It should be understood that the specific embodiments described herein are only used to explain the disclosure and not to limit the disclosure.

The disclosure provides an eAgo protein from eukaryotes, and the eAgo protein is any of the following:

i) a protein with an amino acid sequence as shown in SEQ ID NO: 1. The protein is derived from Thermomycetes thermophilus eukaryotes, named TteAgo protein. A sequence of a nucleic acid molecule encoding the TteAgo protein is shown in SEQ ID NO: 3.

ii) a protein with at least 50% sequence identity with the amino acid sequence as shown in SEQ ID NO: 1 and the same function as the protein with the amino acid sequence as shown in SEQ ID NO: 1; preferably, at least 80% sequence identity with the amino acid sequence as shown in SEQ ID NO: 1; more preferably, at least 90% sequence identity with the amino acid sequence as shown in SEQ ID NO: 1; and most preferably, at least 95% sequence identity with the amino acid sequence as shown in SEQ ID NO: 1. A sequence of nucleic acid molecule encoding this type of protein is: a polynucleotide sequence hybridizing with the DNA molecule shown in SEQ ID NO: 3 under a strict condition, or a nucleotide sequence having at least 50%, at least 80%, at least 90% or at least 95% sequence identity with the sequence shown in SEQ ID NO: 3.

In an embodiment, the eAgo protein has binding activity to a guide RNA (also referred to as gRNA) and a guide single stranded DNA (ssDNA), and has nuclease activity to a target RNA (also referred to as tRNA) and a target DNA (also referred to as tDNA). Therefore, when the guide RNA or the guide ssDNA having most of pairing with the sequence of the target RNA or the target DNA binds to the eAgo protein to form an eAgo complex (also referred to as eAgo-guide complex), and when the eAgo-guide complex binds to the target RNA or the target DNA, site-specific cleavage of the target DNA or the target RNA can occur.

The guide molecule can be 5′-terminal phosphorylated RNA and/or 5′-terminal phosphorylated ssDNA, or hydroxylated RNA and/or hydroxylated ssDNA. The guide molecule can contain 5′-terminal-triphosphate.

In an embodiment, a length of the guide ssDNA is 12 to 30 nucleotides, preferably 15 to 20 nucleotides, such as 16, 17 or 18 nucleotides.

In an embodiment, the eAgo protein has nuclease activity in a temperature range of 25-65° C.; advantageously and preferably, the eAgo protein of the disclosure has the nuclease activity at 37° C.

In an embodiment, the nuclease activity of the eAgo protein requires the presence of cations, which are any one or any combination selected from the group consisting of Fe²⁺, Co²⁺, Ni²⁺, Cu²⁺, Zn²⁺, Mg²⁺, Mn²⁺, and Ca²⁺. Preferably, the cations are Mn²⁺ and Mg²⁺. A concentration range of the cations can vary from about 0.01 millimoles per liter (mM) to about 2000 mM; more preferably, the concentration range is from about 0.05 mM to about 20 mM.

In some embodiments, the N-terminal and/or C-terminal of the eAgo protein have multiple nuclear localization sequences (NLS).

In some embodiments, the target RNA has no advanced structure. In other embodiments, the target RNA has an advanced structure. Other possible target RNAs include a double-stranded RNA, an RNA transcribed in vitro, a viral genome RNA, a messenger RNA (mRNA) and other RNA in the cell.

Specifically, a length of the eAgo protein in the disclosure is 1082 amino acids as shown in SEQ ID NO: 1, or a longer or shorter continuous fragment of amino acids. The number of amino acids (longer or shorter) may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, ... (consecutive digits), and/or 1082.

The above continuous amino acids are defined as functional fragments that include or are less than the total length of the eAgo protein (1082 amino acids) described in the disclosure, but retain the formation of the eAgo-guide complex with the guide molecule and the site-specific cleavage activity on the target RNA and/or the target DNA.

The eAgo protein and the eAgo complex with nuclease activity can specifically cleave the target RNA or the target DNA in vivo or in vitro, and in vivo is intracellular.

The disclosure also provides a method of genetic material for site-specific modification in cells, specifically: introducing an expression vector containing a polynucleotide sequence encoding the eAgo protein into the cell, and simultaneously or not simultaneously introducing one or more guide RNA and/or guide ssDNA, so as to express the eAgo protein in the cell.

In some embodiments, the site-specific modification method occurs in isolated cells. In other embodiments, the method in the disclosure may occur in situ cells, which may be living tissues, organs or animals including humans.

The eAgo protein in the site-specific modification method may be encoded by an expression vector. In other such methods, one or more eAgo proteins may be encoded by two expression vectors. In some embodiments, one expression vector can encode all eAgos proteins.

The expression vector in the site-specific modification method may be contained in a viral vector, such as a lentivirus vector or a retrovirus vector.

The site-specific modification method may be used in eukaryotic cells.

The kit provided by the disclosure include the following three types:
kit 1, including: the eAgo protein described in the disclosure, and a guide RNA and/or a guide ssDNA;
kit 2, including: an expression vector containing a polynucleotide sequence encoding the eAgo protein, and the guide RNA and/or the guide ssDNA; and
kit 3, including: a virus vector containing the expression vector, and a virus vector encoding the guide RNA and/or the guide ssDNA.

When the eAgo protein of the disclosure has the binding activity to the guide RNA or the guide ssDNA, but has no nuclease activity to the target DNA and the target RNA, the guide RNA or the guide ssDNA having most of pairing with the target RNA or the target DNA binds to the eAgo protein to form the eAgo-guide complex, and when the eAgo-guide complex binds to the target RNA or the target DNA, site-specificity of the target RNA or the target DNA is blocked.

The eAgo protein without nuclease activity can be prepared as follows: a new nuclease activity is created by mutating one or more amino acid residues essential for the catalytic activity of the eAgo protein, especially the loss of endonuclease activity. That is to say, at least one amino acid in the evolutionarily conserved amino acid quadruplet (i.e., DEDD) is mutated. Therefore, the mutation may be a single amino acid change in any one or more of the following amino acid sequences of the TteAgo protein:
FVGYDVTHP, shown in SEQ ID NO: 27;
KSRVEQVGGK, shown in SEQ ID NO: 28;
VIFRDGVSE, shown in SEQ ID NO: 29; and
AYYADLVAA, shown in SEQ ID NO: 30.

More specifically, the amino acid change is a single change at one or more of the highlighted residues. Preferably, a single mutation is a non-conservative substitution, such as from D (i.e., aspartic acid) to A (i.e., alanine), or from E (i.e., glutamic acid) to A. Therefore, any substitution other than D to E or E to D is possible.

In addition to substitution, one or more highlighted residues can be simply deleted. In an embodiment, one or more amino acids in the amino acid sequence can be deleted continuously or discontinuously, or one or more sequence motifs can be deleted as a whole. Any combination of the above changes can be made, for example, a non-conservative change in one motif and an absence of the other three motifs. The structural features of nuclease-deficient eAgo protein in the disclosure can include any structural changes as defined above for the eAgo protein with nuclease activity, such as the sequence identity range compared with the reference sequence, the composition of the eAgo protein in terms of amino acid domain and the total length in terms of amino acid. The definition of the guide is similar to the guide used in the eAgo protein with nuclease activity in the disclosure.

For the eAgo complex without nuclease activity, this means that there is an advantageous method to block specific sites in the target DNA or the RNA through specific sequence recognition. The target can be single-stranded and double-stranded. Such site-specific blocking provides an accurate means of blocking target gene transcription, or blocking, disrupting or interfering with specific sites involved in gene expression regulation.

Therefore, the disclosure provides a method for site-specific targeted blocking of target nucleic acid in cells, including the following steps: mixing an eAgo protein without nuclease activity with a guide RNA or a guide ssDNA to form an eAgo complex; transferring the eAgo complex into the cells (such as through transformation, transfection, fiber injection, etc.), and the guide sequence is substantially complementary to the nucleotide sequence contained in the target nucleic acid.

Based on this, the method for site-specific targeted blocking of target nucleic acid in cells can also adopt the following steps: transfecting, transforming or transducing the cells with the expression vector containing a nucleic acid molecule for encoding the eAgo protein without nuclease activity; transfecting, transforming or transducing a first guide RNA sequence or a first guide ssDNA sequence and a second guide ssRNA sequence or a second guide ssDNA sequence; in which at least one guide molecule sequence is substantially complementary to the nucleotide sequence contained in the target nucleic acid, and the eAgo protein generated by expression in the cell and the guide molecule form the eAgo complex capable of blocking specific sites.

In an embodiment, the method for site-specific blocking of target polynucleotides using the eAgo protein without nuclease activity can be targeted to destroy gene expression and/or the control elements of the gene expression, such as promoters or enhancers.

Among the various methods for site-specific blocking of the target DNA or the target RNA, particularly preferred or optional aspects refer to the eAgo protein without nuclease activity defined in the disclosure.

### Embodiment 1 Expression and Purification of TteAgo Protein

The pET28a-CL7-TteAgo plasmid is transformed into Escherichia coli BL21(DE3), and a single colony is inoculated into a Luria-Bertani (LB) liquid medium containing 50 micrograms per milliliter (µg/mL) kanamycin and cultured in a shake flask at 37° C. and 220 revolutions per minute (rpm). When the optical density at 600 nanometers (OD600) reaches 0.8, the bacteria are moved to a shaker at 18° C. and induced by isopropylthio-β-galactoside (IPTG) overnight. The bacteria are collected by centrifugation at 6000 rpm for 10 minutes (min), washed with Buffer A (including 20 mM Tris-HCl pH 7.4, 500 mM NaCl, and 10 mM imidazole), suspended in the Buffer A, added phenylmethanesulfonyl fluoride (PMSF) at a final concentration of 1 mM, and disrupted under high pressure. Then, the supernatant is collected by centrifugation at 18000 rpm for 30 min. After the supernatant is filtered, nickel-nitrilotriacetic acid (Ni-NTA) purification is performed. An amino acid sequence of CL7-TteAgo fusion protein is shown in SEQ ID NO: 2, and a polynucleotide sequence of CL7-TteAgo fusion protein is shown in SEQ ID NO: 4.

A column is washed with the Buffer A containing 10 mM imidazole (added in three times) for 10 column volumes, then the column is washed with 200 mM imidazole for 5 column volumes, and samples are taken for sodium dodecyl sulfate-polyacrylamide gel electrophoresis (SDS-PAGE) detection. 200 mM imidazole eluant (containing high purity target protein) is collected and incubated with activated agarose beads coupled with Im7 protein. The fusion expressed TteAgo-CL7 fusion protein can be specifically combined with the Im7 protein, specifically combined on the agarose beads, impurity proteins are removed by repeated elution (10 times) of high salt (1 m NaCl) and low salt (100 mM NaCl), and then the pure target protein is obtained by cleavage with 3C protease. The purified protein is collected and identified the purity with SDS-PAGE, and ultrafiltered to Buffer B (including 20 mM Tris-HCl pH 7.4, 500 mM NaCl, and 1 mM TCEP). The protein is divided into small parts and stored at -80° C. after quick freezing with liquid nitrogen.

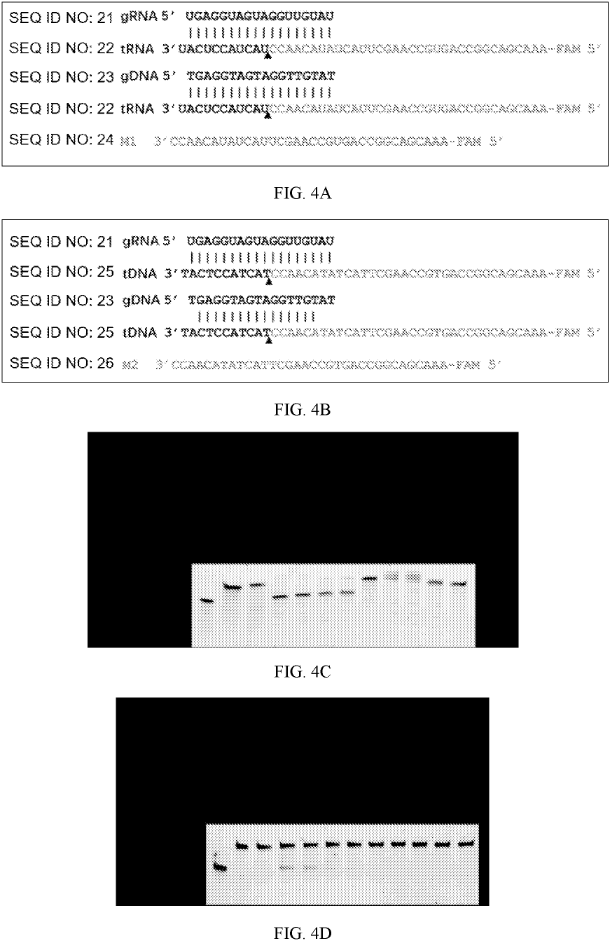

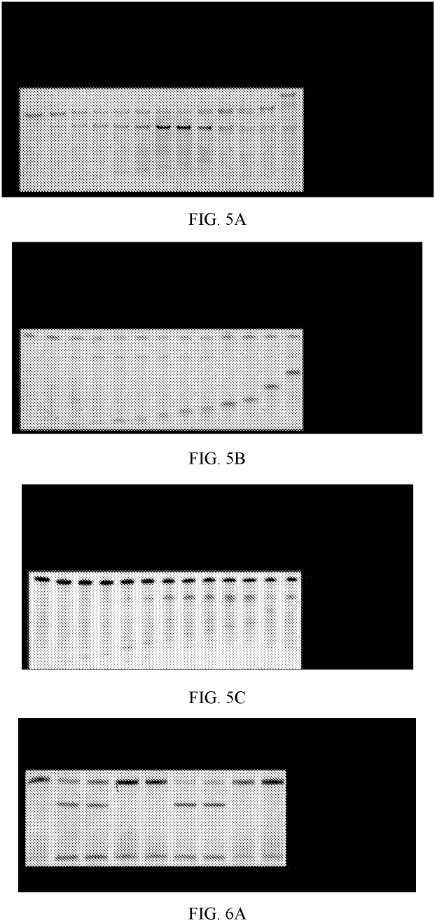

FIG. 2 shows the region in which the catalytic DEDX quadruplet and the sequence identity of TteAgo and other Agos. FIG. 3 shows results of gel analysis of TteAgo after purification of TteAgo by Ni-NTA column and molecular sieve. It is calculated that an expected size of the TteAgo protein is about 118 kilodalton (kDa) based on http://www.expasy.org/.

### Embodiment 2 Cleavage Activity of TteAgo Protein

In order to evaluate which combinations of guide RNA/DNA and target RNA/DNA can be cleaved by the TteAgo protein, the activity of all possible combinations is determined in this embodiment.

The cleavage experiments are carried out at 37° C. with a molar ratio of 5:2:1 (TteAgo: guide: target). 1 uM TteAgo is mixed with 400 nM guide (i.e., guide molecule, e.g., RNA/DNA) in a reaction buffer containing 10 mM HEPES-NaOH (pH 7.5), 100 mM NaCl, 5 mM MnC12 and 5% glycerol, and incubated at 37° C. for 10 min for guide loading. The target nucleic acid (i.e., target RNA/DNA) is added to a final concentration of 200 nM. After reaction at 37° C. for 1 hour, the sample is mixed with 2x RNA loading dyes ( 95% formamide, 18 mM EDTA, 0.025% SDS and 0.025% bromophenol blue) and heated at 95° C. for 5 min to terminate the reaction. The cleavage products are analyzed by 20% denatured Tris-borate EDTA-polyacrylamide gel electrophoresis (TBE-PAGE), stained by SYBR^{®} Gold (Invitrogen), and visualized by GelDoc™ XR+(Bio-Rad).

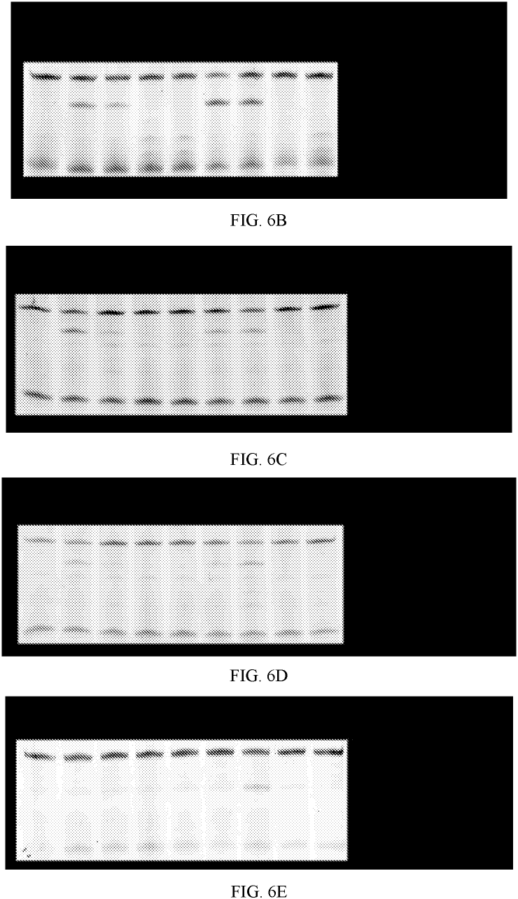

FIGS. 4A-4B are schematic diagrams of guide RNA, guide DNA, target RNA and target DNA used for testing, and arrows indicate predicted cleavage sites. FIGS. 4C-4D are urea/polyacrylamide gel electrophoresis diagrams of products of the TteAgo protein cleaving the target RNA and the target DNA. It can be seen from the diagrams that: a) no product band (34 nucleotide base pairs abbreviated as nt) is observed in the DNA/RNA (guide/target) control assay incubated without the TteAgo, indicating that the formation of the product band is the result of nuclease activity of the TteAgo; b) the TteAgo can cleave the target RNA by using 5′-terminal phosphorylated guide RNA, 5′-terminal hydroxylated guide RNA, 5′-terminal phosphorylated guide DNA and 5′-terminal hydroxylated guide DNA; and c) the TteAgo can cleave the target DNA by using 5′-terminal phosphorylated guide RNA and 5′-terminal hydroxylated guide RNA.

In addition, the first and third amino acids D of the quadruplet DEDD catalyzed by the TteAgo are mutated into amino acid A, and the double mutant DM is recorded as TteAgo-DM. As shown in FIGS. 4C-4D, it can be seen that the TteAgo-DM loses the activity of the guide DNA cleaving the target RNA and the target DNA.

### Embodiment 3 Influence of Length of Guide Molecule on Cleavage Effect

Referring to the experimental method in the embodiment 2, the guide RNA or guide DNA with different length binds to the TteAgo to verify its activity of cleaving the target RNA or the target DNA.

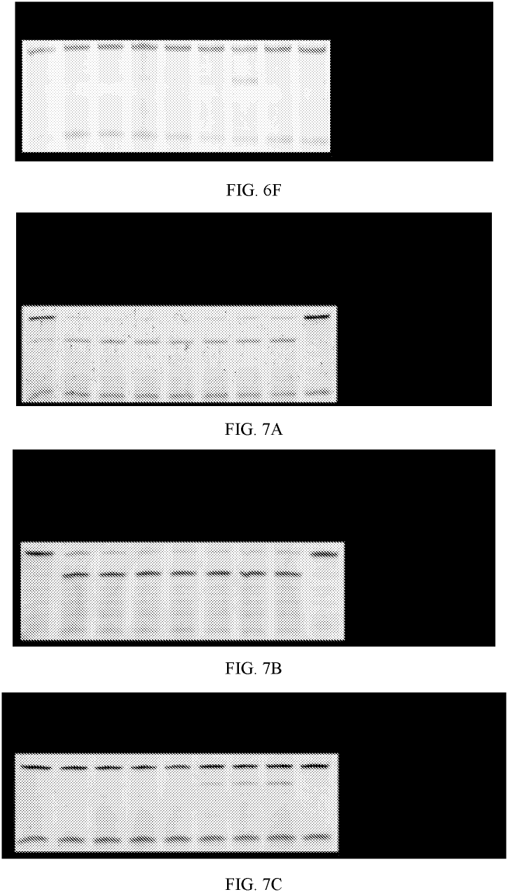

The detection results are shown in FIGS. 5A-5C. FIG. 5A shows that the TteAgo shows guide-guided cleavage of target RNA within 30 min under a guide condition of 5′terminal phosphorylated RNA with the length of 12-30 nt. FIG. 5B shows that the TteAgo shows guide-guided cleavage of target RNA within 30 min under a guide condition of 5′terminal phosphorylated DNA with the length of 12-30 nt. FIG. 5C shows that the TteAgo shows guide-guided cleavage of target ssDNA within 60 min under a guide condition of 5′terminal phosphorylated RNA with the length of 12-30 nt. It can be seen from FIGS. 5A-5C that the target RNA can be effectively cleaved when the length of the guide RNA is in a range of 12-25 nt and the length of the guide DNA is in a range of 12-30 nt; and the target ssDNA can be effectively cleaved when the length of guide RNA is in a range of 12-30 nt.

### Embodiment 4 Influence of Metal Ions on Cleavage Effect

### Influence of Metal Ion Type

Referring to the experimental method in the embodiment 2, different divalent metal ions, including Fe²⁺, Co²⁺, Ni²⁺, Cu²⁺, Zn²⁺, Mg²⁺, Mn²⁺, Ca²⁺, are used in a reaction buffer to verify the effect of cations on the activity of cleaving the target RNA or the target DNA.

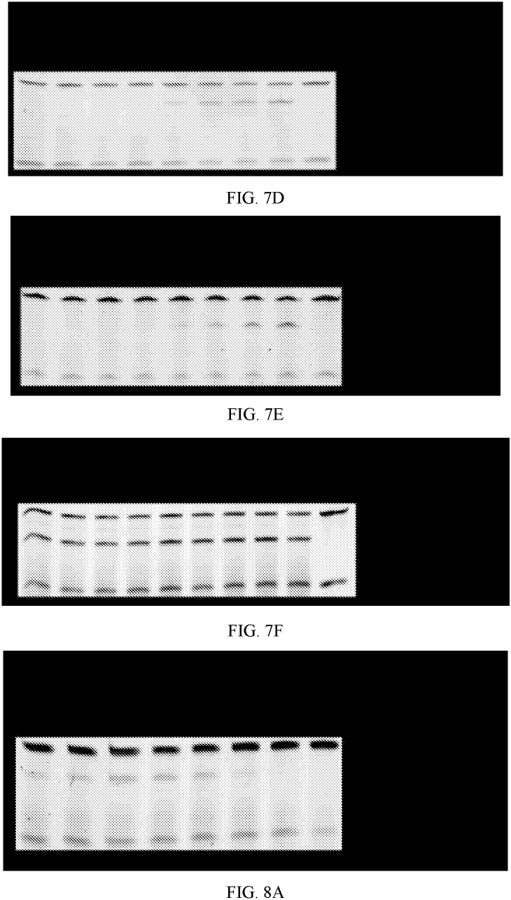

The detection results are as shown in FIGS. 6A-6F. It can be seen that when the cations are Mn²⁺, Mg²⁺, Co²⁺ and/or Ni²⁺, the TteAgo can effectively cleave the target RNA by binding the guide RNA or the guide DNA (FIGS. 6A, 6B, 6C, 6D). When the cation is Mn²⁺, the TteAgo binding the guide RNA can effectively cleave the target DNA (FIGS. 6E-6F).

### Influence of Metal Ion Concentration

Mn²⁺ and Mg²⁺ are selected to find the concentration range of Mn²⁺ or Mg²⁺ in which the TteAgo shows guide-guided cleavage of the target RNA within 15 min.

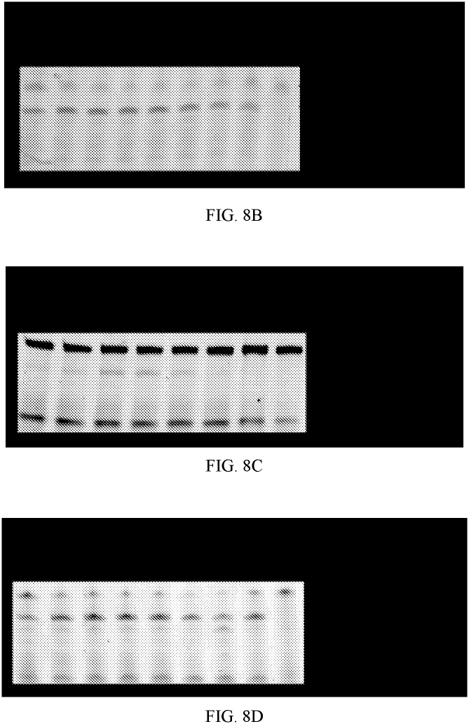

The detection results are shown in FIGS. 7A-7F. When the concentration range of Mn²⁺ and Mg²⁺ is set to 0~10 mM, and the guide is 5′-terminal phosphorylated RNA, when the concentration of Mn²⁺ and Mg²⁺ is in a range of 0.05 mM to 10 mM, the target RNA can be cleaved efficiently (FIGS. 7A-7B). When the concentration range of Mn²⁺ and Mg²⁺ is set to 0~10 mM, and the guide is 5′-terminal phosphorylated DNA, when the concentration of Mn²⁺ is in a range of 2.5 mM to 10 mM, and when the concentration of Mg²⁺ is in a range of 1 mM to 10 mM, the target RNA can be cleaved (FIGS. 7C-7D). When the concentration of Mn²⁺ is in a range of 1 mM to 50 mM and the guide is 5′-terminal phosphorylated RNA, the target DNA can be effectively cleaved (FIGS. 7E-7F).

### Embodiment 5 Influence of Temperature on Cleavage Effect

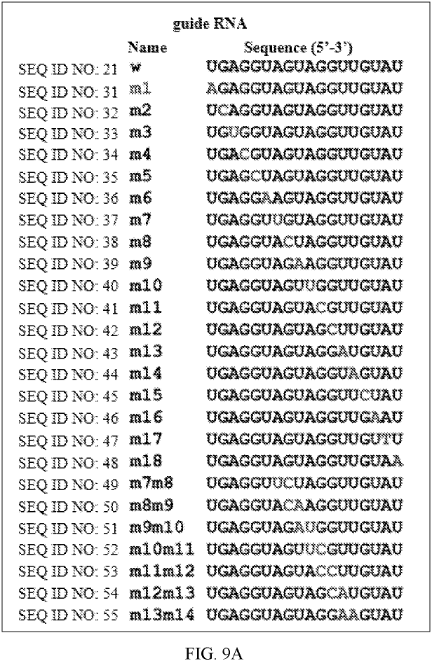

Referring to the experimental method in the embodiment 2, the temperature range at which the TteAgo displays guide-guided cleavage of the target RNA within 15 min is found. As shown in FIGS. 8B and 8D, the 5′-terminal phosphorylated guide RNA and 5′-terminal hydroxylated guide RNA can cleave the target RNA at 25~70° C., preferably 37~60° C.

Using the same method, the temperature range at which the TteAgo displays guide-guided cleavage of the target DNA within 60 min is found. As shown in FIGS. 8A and 8C, 5′-terminal phosphorylated guide RNA and 5′-terminal hydroxylated guide RNA can cleave the target DNA at 30~60° C., preferably 37~45° C.

### Embodiment 6

Referring to the experimental method in the embodiment 2, the influence of single-base or double-base mutation of the guide molecule on the TteAgo cleaving the target RNA or the target DNA is as follows.

### Influence of Single-Base and/or Double-Base Mutations in Guide RNA on TteAgo Cleaving the Target RNA

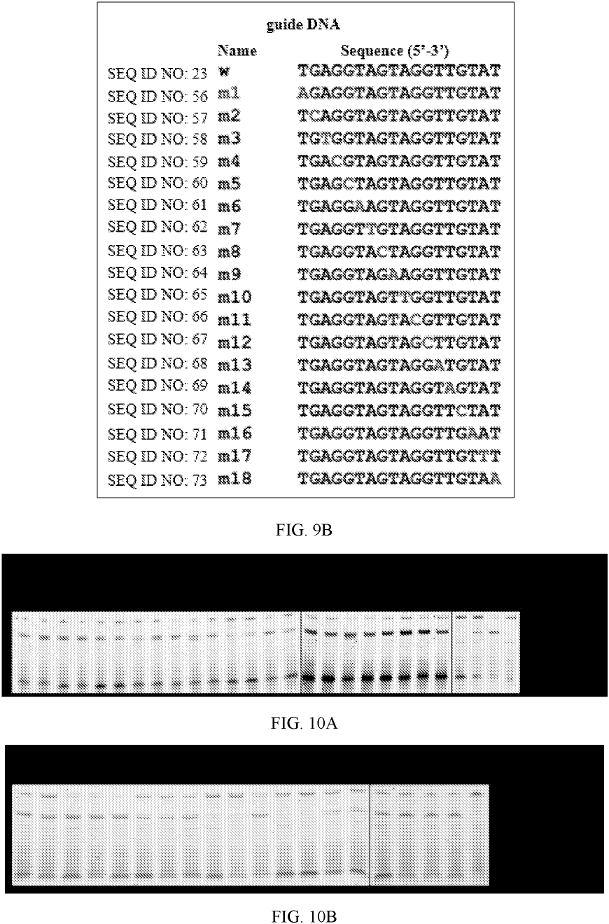

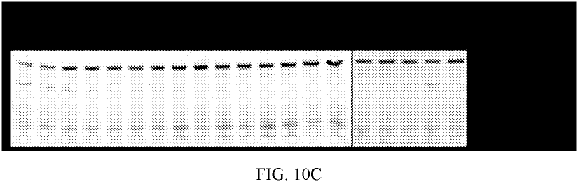

The guide RNAs with single-base or double-base mutation are synthetized (m1 as shown in SEQ ID NO: 31, m2 as shown in SEQ ID NO: 32, m3 as shown in SEQ ID NO: 33, m4 as shown in SEQ ID NO: 34, m5 as shown in SEQ ID NO: 35, m6 as shown in SEQ ID NO: 36, m7 as shown in SEQ ID NO: 37, m8 as shown in SEQ ID NO: 38, m9 as shown in SEQ ID NO: 39, m10 as shown in SEQ ID NO: 40, m11 as shown in SEQ ID NO: 41, m12 as shown in SEQ ID NO: 42, m13 as shown in SEQ ID NO: 43, m14 as shown in SEQ ID NO: 44, m15 as shown in SEQ ID NO: 45, m16 as shown in SEQ ID NO: 46, m17 as shown in SEQ ID NO: 47, m18 as shown in SEQ ID NO: 48, m7m8 as shown in SEQ ID NO: 49, m8m9 as shown in SEQ ID NO: 50, m9m10 as shown in SEQ ID NO: 51, m10m11 as shown in SEQ ID NO: 52, m11m12 as shown in SEQ ID NO: 53, m12m13 as shown in SEQ ID NO: 54, and m13m14 as shown in SEQ ID NO: 55, specific mutations are shown in FIG. 9A), and the above guide RNAs respectively mediate TteAgo to cleave the target RNA. As shown in FIG. 10A, when the 11^{th} and 12^{th} bases of the RNA guide are mutated simultaneously, TteAgo has the weakest cleavage activity to the target RNA.

### Influence of Single-Base Mutation in Guide DNA on TteAgo Cleaving The Target RNA

The guide DNAs with single-base mutation are synthetized (m1 as shown in SEQ ID NO: 56, m2 as shown in SEQ ID NO: 57, m3 as shown in SEQ ID NO: 58, m4 as shown in SEQ ID NO: 59, m5 as shown in SEQ ID NO: 60, m6 as shown in SEQ ID NO: 61, m7 as shown in SEQ ID NO: 62, m8 as shown in SEQ ID NO: 63, m9 as shown in SEQ ID NO: 64, m10 as shown in SEQ ID NO: 65, m11 as shown in SEQ ID NO: 66, m12 as shown in SEQ ID NO: 67, m13 as shown in SEQ ID NO: 68, m14 as shown in SEQ ID NO: 69, m15 as shown in SEQ ID NO: 70, m16 as shown in SEQ ID NO: 71, m17 as shown in SEQ ID NO: 72, and m18 as shown in SEQ ID NO: 73, specific mutations are shown in FIG. 9B), and the above guide DNAs respectively mediate TteAgo to cleave the target RNA. As shown in FIG. 10B, when the guide DNA is mutated at the 8^{th}, 9^{th}, 11^{th} or 12^{th} base, TteAgo has the weakest cleavage activity to the target RNA.

### Influence of Single-Base Mutation in Guide RNA on TteAgo Cleaving Target DNA

The guide RNAs with single-base mutation are synthetized (m1, m2, m3, m4, m5, m6, m7, m8, m9, m10, m11, m12, m13, m14, m15, m16, m17, m18, specific mutations are shown in FIG. 9B), and the above guide RNAs respectively mediate TteAgo to cleave the target DNA. As shown in FIG. 10C, when the single-base mutation occurs at the 3^{rd} to the 17^{th} positions of the guide RNA, the cleavage activity of TteAgo to the target DNA is significantly reduced.

In summary, the eukaryotic Argonaute protein provided by the disclosure has binding activity to the guide RNA and the guide ssDNA, and has nuclease activity to both the target RNA and the target DNA, and the eAgo protein of the disclosure can carry out site-specific modification on intracellular and extracellular genetic material. Therefore, it can be effectively applied in many fields of biotechnology, such as nucleic acid detection, gene editing and gene modification.

The above description is only specific embodiments of the disclosure, but the scope of protection of the disclosure is not limited thereto. Any modification, equivalent substitution and improvement made by any person skilled in the art within the technical scope disclosed by the disclosure within the spirit and principles of the disclosure shall be included by the scope of protection of the disclosure.

What is claimed is:

1. An application of a eukaryotic Argonaute (eAgo) complex, comprising:
specifically cleaving a target nucleic acid in vitro by using the eAgo complex;
wherein the eAgo complex is formed by a combination of an Argonaute protein and a guide molecule;
wherein the Argonaute protein has a nuclease activity, and an amino acid sequence of the Argonaute protein is shown in SEQ ID NO: 1;
wherein a nucleotide sequence of a nucleic acid molecule encoding the Argonaute protein is shown in SEQ ID NO: 3; and
wherein the guide molecule is one of a guide ssDNA and a guide RNA selected from a group consisting of a 5′-terminal phosphorylated guide RNA, a 5′-terminal hydroxylated guide RNA, a 5′-terminal phosphorylated guide ssDNA, and a 5′-terminal hydroxylated guide ssDNA; and a length of the guide ssDNA is in a range of 12 to 30 nucleotides.

2. The application according to claim 1, specifically comprising:
making the eAgo complex contact with the target nucleic acid to specifically cleave the target nucleic acid by the eAgo complex, wherein the target nucleic acid contains a nucleotide sequence complementary to at least 12 bases of the guide molecule.

3. The application according to claim 1, wherein the Argonaute protein has the nuclease activity at a temperature in a range of 10~65 Celsius degree (°C).

4. The application according to claim 1, wherein the Argonaute protein has the nuclease activity in a solution of bivalent metal cations, and the bivalent metal cations are at least one selected from a group consisting of Fe²⁺, Co²⁺, Ni²⁺, Cu²⁺, Zn²⁺, Mg²⁺, Mn²⁺, and Ca²⁺.

5. The application according to claim 4, wherein the divalent metal cations are at least one of Mn²⁺ and Mg²⁺.

6. The application according to claim 1, wherein the nuclease activity of the Argonaute protein has at least one of single-base specificity and double-base specificity.


SEQUENCE LISTING (73 sequences)

SEQ ID NO: 1 - protein, 1082 aa, synthetic construct
MSDSRGRGYGGGRGGGRGGRGGQAGEGFRGGGGRGGYPGGGRGGGDGYRGGRGGGGGEFRGGRGGRGDFRGGRGGGGRGGRGGYGGSGPDVFLGAGPSIPAPDAAVTELEDRWIEKHGIQSRTTGASELESKMADLSLGTISMPKRPGFGTGGNPVVLWANYFNVNLKLGAVYRYDLRVISKKLTKEQDDALSKQQESASKKAKGKPKQASGQQPNAPKDAREAKGKKLSEVIKLALDRLPGEPAIATEYKQQLVTTEKLQVPPDGLMQVELAEPGRNPETWYVRFDGPSSINIAGLMDYVRSLEDKNDGVFPKFPEEIDALGIVLGHTARANLNTAAIGSSRFFAIDQARKDQASMPPDSRIEILRGYVQSVRPATGRLLLNTNVTHAVFRKAVKLDELFQKCGLANLHLPQQRPNHALRTLDGLNKFLAKSRIECKVPGERPGEFFKIQRGMAGLATTKDGKDEDQKPEFTESGFRFGTPATVRFYLRKPKDLGAKPPPGLSFDTMVLVSDYYKARYGIRADPGLPLINVGTAGKPIYILAEFCTLLPGQPLKARLSPQEQDAMIRFACRPPPENALSVTTSARELLALDNNMLLDKFGITVDKHLITVKGRELPPPAVGYLRGNSIERVTPENGGWLMKGVKVCKSGRRIANWAFLVIGKARPPIDFGTIKSAVGGFARFLNNNMGIDMNMQPVPANGYQTAGTSEEDLRNAFRTISKQKPQPEFILVLLPDKDATTYNIVKKLGDVEYGITTVCVRQEMLTKEQGQMGYFANVGLKVNLKFGGINHRVRDETGLVDKTMFVGYDVTHPTNLPGGAGDNAPSLVGLVASVDNSLAQWPAVTWENKSRVEQVGGKTDEGQFIAHFKDRLRLWQKHNSNRLPENIVIFRDGVSEGQFSMVLEKELPNIRQACQETYPARPNAQPRLSLIVSVKRHQTRFYPTDRNHIHPRSKSPKEGTVVDRGVTNVRYWDFFLQAHASLQGTARPAHYTVLLDEIFRHKFGPNAADALETLTHNMCYAYGRATKAVSICPPAYYADLVAARARIHKSELFENVQSLASSEQSSVSRRKVHDRLKDTMYYI

SEQ ID NO: 2 - protein, 1231 aa, synthetic construct
MHHHHHHQQRPSKSNEPGKATGEGKPVNNKWLNNAGKDLGSPVPDRIANKLRDKEFESFDDFRETFWEEVSKDPELSKQFSRNNNDRMKVGKAPKTRTQDVSGKRTSFELNHQKPIEQNGGVYDMDNISVVTPKRNIDIEGLEVLFQGPMSDSRGRGYGGGRGGGRGGRGGQAGEGFRGGGGRGGYPGGGRGGGDGYRGGRGGGGGEFRGGRGGRGDFRGGRGGGGRGGRGGYGGSGPDVFLGAGPSIPAPDAAVTELEDRWIEKHGIQSRTTGASELESKMADLSLGTISMPKRPGFGTGGNPVVLWANYFNVNLKLGAVYRYDLRVISKKLTKEQDDALSKQQESASKKAKGKPKQASGQQPNAPKDAREAKGKKLSEVIKLALDRLPGEPAIATEYKQQLVTTEKLQVPPDGLMQVELAEPGRNPETWYVRFDGPSSINIAGLMDYVRSLEDKNDGVFPKFPEEIDALGIVLGHTARANLNTAAIGSSRFFAIDQARKDQASMPPDSRIEILRGYVQSVRPATGRLLLNTNVTHAVFRKAVKLDELFQKCGLANLHLPQQRPNHALRTLDGLNKFLAKSRIECKVPGERPGEFFKIQRGMAGLATTKDGKDEDQKPEFTESGFRFGTPATVRFYLRKPKDLGAKPPPGLSFDTMVLVSDYYKARYGIRADPGLPLINVGTAGKPIYILAEFCTLLPGQPLKARLSPQEQDAMIRFACRPPPENALSVTTSARELLALDNNMLLDKFGITVDKHLITVKGRELPPPAVGYLRGNSIERVTPENGGWLMKGVKVCKSGRRIANWAFLVIGKARPPIDFGTIKSAVGGFARFLNNNMGIDMNMQPVPANGYQTAGTSEEDLRNAFRTISKQKPQPEFILVLLPDKDATTYNIVKKLGDVEYGITTVCVRQEMLTKEQGQMGYFANVGLKVNLKFGGINHRVRDETGLVDKTMFVGYDVTHPTNLPGGAGDNAPSLVGLVASVDNSLAQWPAVTWENKSRVEQVGGKTDEGQFIAHFKDRLRLWQKHNSNRLPENIVIFRDGVSEGQFSMVLEKELPNIRQACQETYPARPNAQPRLSLIVSVKRHQTRFYPTDRNHIHPRSKSPKEGTVVDRGVTNVRYWDFFLQAHASLQGTARPAHYTVLLDEIFRHKFGPNAADALETLTHNMCYAYGRATKAVSICPPAYYADLVAARARIHKSELFENVQSLASSEQSSVSRRKVHDRLKDTMYYI

SEQ ID NO: 3 - DNA, 3246 bp, synthetic construct
  source 1..3246: mol_type = other DNA
atgagcgatagccgtggtcgtggttatggtggtggtcgcggtggtggccgtggcggtcgtggtggtcaagccggtgaaggttttcgcggtggcggtggacgtggtggttatcctggcggaggacgcggaggtggtgatggctatcgcggaggtagaggtggtggcggaggtgaatttcgtggcggaagaggtggtagaggcgattttagaggcggacgaggcggaggcggtcgcggaggtcgtggtggatatggtggtagcggtccggatgtttttttaggtgcaggtccgagcattccggcaccggatgcagcagttaccgaactggaagatcgttggattgaaaaacatggtattcagagccgtaccaccggtgcaagtgaactggaaagcaaaatggcagatctgagcctgggcaccattagcatgccgaaacgtcctggttttggtacaggtggtaatccggttgttctgtgggcaaattactttaacgtgaatctgaaactgggtgccgtgtatcgttatgatctgcgtgttattagcaagaaactgaccaaagaacaggatgatgcactgagcaaacagcaagaaagcgcaagcaaaaaagcaaaaggtaaaccgaaacaggcaagcggtcagcagccgaatgcaccgaaagatgcacgtgaagccaaaggcaaaaaactgagcgaagttattaaactggcactggatcgtctgcctggtgaaccggcaattgcaaccgaatataaacagcagctggttaccaccgaaaaactgcaggttccgcctgatggtctgatgcaggttgaactggcagaaccgggtcgtaatccggaaacctggtatgttcgttttgatggtccgagtagcattaatatcgcaggtctgatggattatgttcgtagcctggaagataaaaacgatggtgtgtttccgaaattcccggaagaaattgatgccctgggtattgttctgggtcataccgcacgtgcaaatctgaatacagcagcaattggtagcagccgtttttttgcaattgatcaggcacgtaaagatcaggcaagcatgccaccggatagccgtattgaaattctgcgtggctatgttcagagcgttcgtccggcaaccggtcgtctgctgctgaatacaaatgttacccatgccgtttttcgtaaagccgttaaactggatgaactgtttcagaaatgtggtctggcaaatctgcatctgccgcagcagcgtccgaatcatgcactgcgtaccctggatggcctgaacaaatttctggcaaaaagtcgcattgaatgcaaagttccgggtgaacgccctggtgaatttttcaaaatccagcgtggtatggcaggtctggccaccacaaaagatggtaaagatgaagatcagaagccggaatttaccgaaagcggttttcgctttggtacaccggcaacagttcgcttttatctgcgtaaacctaaagatctgggtgcaaaaccgcctcctggtctgagctttgataccatggttctggttagcgattattacaaagcccgttatggtattcgtgcagatccgggtctgccgctgattaatgttggcaccgcaggcaaaccgatttatatcctggcagaattttgtaccctgctgcctggtcagccgctgaaagcacgtctgagtccgcaagagcaggatgcaatgattcgttttgcatgtcgtccgccacctgaaaatgccctgagcgttaccacctcagcacgtgaactgctggccctggataataacatgctgctggataaatttggcatcaccgttgataaacatctgattaccgttaaaggtcgcgaactgcctcctccggcagttggctatctgcgtggtaatagtattgaacgtgttacaccggaaaatggtggttggctgatgaaaggtgttaaagtttgtaaaagcggtcgtcgtattgcaaattgggcatttctggttattggtaaagcccgtccgcctattgattttggtacgattaaaagcgcagttggtggttttgcccgttttctgaataacaatatgggcatcgatatgaacatgcagccggttccggcaaatggttatcagaccgcaggtacaagcgaagaagatctgcgcaacgcatttcgtaccattagtaaacagaaaccgcagcctgaatttattctggttctgctgccagataaagatgcaaccacctataacatcgtgaaaaaactgggagatgtggaatatggtattaccaccgtttgtgttcgtcaagaaatgcttacaaaagaacaaggtcagatgggctattttgcaaatgttggtctgaaagtgaacctgaaatttggtggcattaatcatcgtgttcgtgatgaaaccggtctggttgataaaaccatgtttgttggttatgatgtgacccatccgaccaatctgccaggtggtgccggtgataatgcaccgtcactggttggtctggtggcaagcgttgataattcactggcacagtggcctgcagttacctgggaaaacaaaagccgtgttgaacaggttggtggtaaaaccgatgaaggccagtttattgcccattttaaagatcgcctgcgtctgtggcagaaacataatagcaatcgcctgccggaaaacattgtgatttttcgtgatggtgttagcgaaggtcagtttagcatggtgctggaaaaagaactgccgaacattcgtcaggcatgtcaagaaacctatccggcacgtccgaatgcgcagcctcgtctgtcactgattgttagcgttaaacgtcatcagacccgtttctatccgaccgatcgtaatcatattcatccgcgtagcaaaagcccgaaagaaggcaccgttgttgatcgtggtgttaccaatgttcgttattgggatttttttctgcaggcacatgcaagcctgcagggcaccgcacgtccggcacattataccgttctgctggacgaaatttttcgccataaattcggtccgaacgcagcagatgcactggaaaccctgacacataatatgtgctatgcctatggtcgtgcaaccaaagcagttagcatttgtccgcctgcatattatgccgatctggttgcagcacgtgcccgtattcataaatcagaactgtttgaaaacgttcagagcctggcaagcagcgaacagagcagcgttagccgtcgtaaagttcatgatcgtctgaaagataccatgtattatatc

SEQ ID NO: 4 - DNA, 3696 bp, synthetic construct
  source 1..3696: mol_type = other DNA
atgcaccatcatcatcatcatcagcagcggccaagcaaaagcaatgaaccgggtaaagcaaccggtgaaggtaaaccggttaataacaaatggctgaacaatgccggtaaagatctgggtagtccggttccggatcgtattgcaaataaactgcgtgataaagaattcgagagcttcgatgattttcgtgaaaccttttgggaagaagttagcaaagatcctgaactgagcaaacagtttagccgcaataacaatgatcgtatgaaagttggtaaagcaccgaaaacacgtacccaggatgttagcggtaaacgtacctcatttgaactgaatcatcagaaaccgattgaacagaatggtggcgtttatgatatggataacattagcgttgttaccccgaaacgcaacattgatattgaaggcttggaggttttgttccagggtccaatgagcgatagccgtggtcgtggttatggtggtggtcgcggtggtggccgtggcggtcgtggtggtcaagccggtgaaggttttcgcggtggcggtggacgtggtggttatcctggcggaggacgcggaggtggtgatggctatcgcggaggtagaggtggtggcggaggtgaatttcgtggcggaagaggtggtagaggcgattttagaggcggacgaggcggaggcggtcgcggaggtcgtggtggatatggtggtagcggtccggatgtttttttaggtgcaggtccgagcattccggcaccggatgcagcagttaccgaactggaagatcgttggattgaaaaacatggtattcagagccgtaccaccggtgcaagtgaactggaaagcaaaatggcagatctgagcctgggcaccattagcatgccgaaacgtcctggttttggtacaggtggtaatccggttgttctgtgggcaaattactttaacgtgaatctgaaactgggtgccgtgtatcgttatgatctgcgtgttattagcaagaaactgaccaaagaacaggatgatgcactgagcaaacagcaagaaagcgcaagcaaaaaagcaaaaggtaaaccgaaacaggcaagcggtcagcagccgaatgcaccgaaagatgcacgtgaagccaaaggcaaaaaactgagcgaagttattaaactggcactggatcgtctgcctggtgaaccggcaattgcaaccgaatataaacagcagctggttaccaccgaaaaactgcaggttccgcctgatggtctgatgcaggttgaactggcagaaccgggtcgtaatccggaaacctggtatgttcgttttgatggtccgagtagcattaatatcgcaggtctgatggattatgttcgtagcctggaagataaaaacgatggtgtgtttccgaaattcccggaagaaattgatgccctgggtattgttctgggtcataccgcacgtgcaaatctgaatacagcagcaattggtagcagccgtttttttgcaattgatcaggcacgtaaagatcaggcaagcatgccaccggatagccgtattgaaattctgcgtggctatgttcagagcgttcgtccggcaaccggtcgtctgctgctgaatacaaatgttacccatgccgtttttcgtaaagccgttaaactggatgaactgtttcagaaatgtggtctggcaaatctgcatctgccgcagcagcgtccgaatcatgcactgcgtaccctggatggcctgaacaaatttctggcaaaaagtcgcattgaatgcaaagttccgggtgaacgccctggtgaatttttcaaaatccagcgtggtatggcaggtctggccaccacaaaagatggtaaagatgaagatcagaagccggaatttaccgaaagcggttttcgctttggtacaccggcaacagttcgcttttatctgcgtaaacctaaagatctgggtgcaaaaccgcctcctggtctgagctttgataccatggttctggttagcgattattacaaagcccgttatggtattcgtgcagatccgggtctgccgctgattaatgttggcaccgcaggcaaaccgatttatatcctggcagaattttgtaccctgctgcctggtcagccgctgaaagcacgtctgagtccgcaagagcaggatgcaatgattcgttttgcatgtcgtccgccacctgaaaatgccctgagcgttaccacctcagcacgtgaactgctggccctggataataacatgctgctggataaatttggcatcaccgttgataaacatctgattaccgttaaaggtcgcgaactgcctcctccggcagttggctatctgcgtggtaatagtattgaacgtgttacaccggaaaatggtggttggctgatgaaaggtgttaaagtttgtaaaagcggtcgtcgtattgcaaattgggcatttctggttattggtaaagcccgtccgcctattgattttggtacgattaaaagcgcagttggtggttttgcccgttttctgaataacaatatgggcatcgatatgaacatgcagccggttccggcaaatggttatcagaccgcaggtacaagcgaagaagatctgcgcaacgcatttcgtaccattagtaaacagaaaccgcagcctgaatttattctggttctgctgccagataaagatgcaaccacctataacatcgtgaaaaaactgggagatgtggaatatggtattaccaccgtttgtgttcgtcaagaaatgcttacaaaagaacaaggtcagatgggctattttgcaaatgttggtctgaaagtgaacctgaaatttggtggcattaatcatcgtgttcgtgatgaaaccggtctggttgataaaaccatgtttgttggttatgatgtgacccatccgaccaatctgccaggtggtgccggtgataatgcaccgtcactggttggtctggtggcaagcgttgataattcactggcacagtggcctgcagttacctgggaaaacaaaagccgtgttgaacaggttggtggtaaaaccgatgaaggccagtttattgcccattttaaagatcgcctgcgtctgtggcagaaacataatagcaatcgcctgccggaaaacattgtgatttttcgtgatggtgttagcgaaggtcagtttagcatggtgctggaaaaagaactgccgaacattcgtcaggcatgtcaagaaacctatccggcacgtccgaatgcgcagcctcgtctgtcactgattgttagcgttaaacgtcatcagacccgtttctatccgaccgatcgtaatcatattcatccgcgtagcaaaagcccgaaagaaggcaccgttgttgatcgtggtgttaccaatgttcgttattgggatttttttctgcaggcacatgcaagcctgcagggcaccgcacgtccggcacattataccgttctgctggacgaaatttttcgccataaattcggtccgaacgcagcagatgcactggaaaccctgacacataatatgtgctatgcctatggtcgtgcaaccaaagcagttagcatttgtccgcctgcatattatgccgatctggttgcagcacgtgcccgtattcataaatcagaactgtttgaaaacgttcagagcctggcaagcagcgaacagagcagcgttagccgtcgtaaagttcatgatcgtctgaaagataccatgtattatatctga

SEQ ID NO: 5 - protein, 36 aa, synthetic construct
YIGIDLSHDELNEKMNLDFILRDGRFILHIANKVAL

SEQ ID NO: 6 - protein, 36 aa, synthetic construct
IVGLDVSRRIDGEILPEHLIHRDGLFPTYYADKIST

SEQ ID NO: 7 - protein, 37 aa, synthetic construct
IIGIDVAPMEQRGESVDMNLLLRDGRITVHYAHKFAN

SEQ ID NO: 8 - protein, 36 aa, synthetic construct
IIGEDTGTNQRGETESGQLLMRDGLVQLHLADRSSK

SEQ ID NO: 9 - protein, 36 aa, synthetic construct
FIGLDVGTRQSGEKIAETVIHRDGFSRTGYADKICK

SEQ ID NO: 10 - protein, 36 aa, synthetic construct; identical to SEQ ID NO: 9

SEQ ID NO: 11 - protein, 36 aa, synthetic construct
IMGLDTGLGAPGERLHLPLFLRDGEIQIHYADKFVK

SEQ ID NO: 12 - protein, 36 aa, synthetic construct
AVGFDAGGRQAGERIPQELLLRDGRVPLHLADRLVK

SEQ ID NO: 13 - protein, 36 aa, synthetic construct
FIGIDVSRSQLGEKLQSTVIHRDGFMNTAYADQAST

SEQ ID NO: 14 - protein, 35 aa, synthetic construct
FIGIDVSHLAGEKIDDTTIHRDGFWRIHYADLSAT

SEQ ID NO: 15 - protein, 35 aa, synthetic construct
YIGLDVCRQSGEKIQINVFHRDGINRTYYADLSSI

SEQ ID NO: 16 - protein, 36 aa, synthetic construct
FVGLDVGTRQNGEKINTEVIHRDGFSRTGYADKICK

SEQ ID NO: 17 - protein, 37 aa, synthetic construct
VLGSDVTHYDGPGEEIITNMYFRDGVSVVYYADLLCT

SEQ ID NO: 18 - protein, 36 aa, synthetic construct
FLGADVTHPQHRQEIIQDIFYRDGVSEAYYAHLVAF

SEQ ID NO: 19 - protein, 37 aa, synthetic construct
FVGYDVTHPKSRVEQVGGKVIFRDGVSEAYYADLVAA

SEQ ID NO: 20 - protein, 37 aa, synthetic construct
FVGYAVTHPKSRVEQVGGKVIFRAGVSEAYYADLVAA

SEQ ID NO: 21 - RNA, 18 nt, synthetic construct
  source 1..18: mol_type = other RNA
tgaggtagtaggttgtat

SEQ ID NO: 22 - RNA, 45 nt, synthetic construct
  source 1..45: mol_type = other RNA
tactccatcatccaacatatcattcgaaccgtgaccggcagcaaa

SEQ ID NO: 23 - DNA, 18 bp, synthetic construct
  source 1..18: mol_type = other DNA
tgaggtagtaggttgtat

SEQ ID NO: 24 - DNA, 34 bp, synthetic construct
  source 1..34: mol_type = other DNA
ccaacatatcattcgaaccgtgaccggcagcaaa

SEQ ID NO: 25 - DNA, 45 bp, synthetic construct
  source 1..45: mol_type = other DNA
tactccatcatccaacatatcattcgaaccgtgaccggcagcaaa

SEQ ID NO: 26 - DNA, 34 bp, synthetic construct; identical to SEQ ID NO: 24
  source 1..34: mol_type = other DNA

SEQ ID NO: 27 - protein, 9 aa, synthetic construct
FVGYDVTHP

SEQ ID NO: 28 - protein, 10 aa, synthetic construct
KSRVEQVGGK

SEQ ID NO: 29 - protein, 9 aa, synthetic construct
VIFRDGVSE

SEQ ID NO: 30 - protein, 9 aa, synthetic construct
AYYADLVAA

SEQ ID NO: 31 - RNA, 18 nt, synthetic construct
  source 1..18: mol_type = other RNA
agaggtagtaggttgtat

SEQ ID NO: 32 - RNA, 18 nt, synthetic construct
  source 1..18: mol_type = other RNA
tcaggtagtaggttgtat

SEQ ID NO: 33 - RNA, 18 nt, synthetic construct
  source 1..18: mol_type = other RNA
tgtggtagtaggttgtat

SEQ ID NO: 34 - RNA, 18 nt, synthetic construct
  source 1..18: mol_type = other RNA
tgacgtagtaggttgtat

SEQ ID NO: 35 - RNA, 18 nt, synthetic construct
  source 1..18: mol_type = other RNA
tgagctagtaggttgtat

SEQ ID NO: 36 - RNA, 18 nt, synthetic construct
  source 1..18: mol_type = other RNA
tgaggaagtaggttgtat

SEQ ID NO: 37 - RNA, 18 nt, synthetic construct
  source 1..18: mol_type = other RNA
tgaggttgtaggttgtat

SEQ ID NO: 38 - RNA, 18 nt, synthetic construct
  source 1..18: mol_type = other RNA
tgaggtactaggttgtat

SEQ ID NO: 39 - RNA, 18 nt, synthetic construct
  source 1..18: mol_type = other RNA
tgaggtagaaggttgtat

SEQ ID NO: 40 - RNA, 18 nt, synthetic construct
  source 1..18: mol_type = other RNA
tgaggtagttggttgtat

SEQ ID NO: 41 - RNA, 18 nt, synthetic construct
  source 1..18: mol_type = other RNA
tgaggtagtacgttgtat

SEQ ID NO: 42 - RNA, 18 nt, synthetic construct
  source 1..18: mol_type = other RNA
tgaggtagtagcttgtat

SEQ ID NO: 43 - RNA, 18 nt, synthetic construct
  source 1..18: mol_type = other RNA
tgaggtagtaggatgtat

SEQ ID NO: 44 - RNA, 18 nt, synthetic construct
  source 1..18: mol_type = other RNA
tgaggtagtaggtagtat

SEQ ID NO: 45 - RNA, 18 nt, synthetic construct
  source 1..18: mol_type = other RNA
tgaggtagtaggttctat

SEQ ID NO: 46 - RNA, 18 nt, synthetic construct
  source 1..18: mol_type = other RNA
tgaggtagtaggttgaat

SEQ ID NO: 47 - RNA, 18 nt, synthetic construct
  source 1..18: mol_type = other RNA
tgaggtagtaggttgttt

SEQ ID NO: 48 - RNA, 18 nt, synthetic construct
  source 1..18: mol_type = other RNA
tgaggtagtaggttgtaa

SEQ ID NO: 49 - RNA, 18 nt, synthetic construct
  source 1..18: mol_type = other RNA
tgaggttctaggttgtat

SEQ ID NO: 50 - RNA, 18 nt, synthetic construct
  source 1..18: mol_type = other RNA
tgaggtacaaggttgtat

SEQ ID NO: 51 - RNA, 18 nt, synthetic construct
  source 1..18: mol_type = other RNA
tgaggtagatggttgtat

SEQ ID NO: 52 - RNA, 18 nt, synthetic construct
  source 1..18: mol_type = other RNA
tgaggtagttcgttgtat

SEQ ID NO: 53 - RNA, 18 nt, synthetic construct
  source 1..18: mol_type = other RNA
tgaggtagtaccttgtat

SEQ ID NO: 54 - RNA, 18 nt, synthetic construct
  source 1..18: mol_type = other RNA
tgaggtagtagcatgtat

SEQ ID NO: 55 - RNA, 18 nt, synthetic construct
  source 1..18: mol_type = other RNA
tgaggtagtaggaagtat

SEQ ID NO: 56 - DNA, 18 bp, synthetic construct
  source 1..18: mol_type = other DNA
agaggtagtaggttgtat

SEQ ID NO: 57 - DNA, 18 bp, synthetic construct
  source 1..18: mol_type = other DNA
tcaggtagtaggttgtat

SEQ ID NO: 58 - DNA, 18 bp, synthetic construct
  source 1..18: mol_type = other DNA
tgtggtagtaggttgtat

SEQ ID NO: 59 - DNA, 18 bp, synthetic construct
  source 1..18: mol_type = other DNA
tgacgtagtaggttgtat

SEQ ID NO: 60 - DNA, 18 bp, synthetic construct
  source 1..18: mol_type = other DNA
tgagctagtaggttgtat

SEQ ID NO: 61 - DNA, 18 bp, synthetic construct
  source 1..18: mol_type = other DNA
tgaggaagtaggttgtat

SEQ ID NO: 62 - DNA, 18 bp, synthetic construct
  source 1..18: mol_type = other DNA
tgaggttgtaggttgtat

SEQ ID NO: 63 - DNA, 18 bp, synthetic construct
  source 1..18: mol_type = other DNA
tgaggtactaggttgtat

SEQ ID NO: 64 - DNA, 18 bp, synthetic construct
  source 1..18: mol_type = other DNA
tgaggtagaaggttgtat

SEQ ID NO: 65 - DNA, 18 bp, synthetic construct
  source 1..18: mol_type = other DNA
tgaggtagttggttgtat

SEQ ID NO: 66 - DNA, 18 bp, synthetic construct
  source 1..18: mol_type = other DNA
tgaggtagtacgttgtat

SEQ ID NO: 67 - DNA, 18 bp, synthetic construct
  source 1..18: mol_type = other DNA
tgaggtagtagcttgtat

SEQ ID NO: 68 - DNA, 18 bp, synthetic construct
  source 1..18: mol_type = other DNA
tgaggtagtaggatgtat

SEQ ID NO: 69 - DNA, 18 bp, synthetic construct
  source 1..18: mol_type = other DNA
tgaggtagtaggtagtat

SEQ ID NO: 70 - DNA, 18 bp, synthetic construct
  source 1..18: mol_type = other DNA
tgaggtagtaggttctat

SEQ ID NO: 71 - DNA, 18 bp, synthetic construct
  source 1..18: mol_type = other DNA
tgaggtagtaggttgaat

SEQ ID NO: 72 - DNA, 18 bp, synthetic construct
  source 1..18: mol_type = other DNA
tgaggtagtaggttgttt

SEQ ID NO: 73 - DNA, 18 bp, synthetic construct
  source 1..18: mol_type = other DNA
tgaggtagtaggttgtaa